(19) United States
(12) Patent Application Publication
(10) Pub. No.: US 2026/0014329 A1
(43) Pub. Date: Jan. 15, 2026

(54) MEDICAL SYSTEMS, DEVICES, AND ASSEMBLIES FOR THE DELIVERY OF A FLUID

(71) Applicant: Boston Scientific Medical Device Limited, Galway (IE)

(72) Inventor: Deepak Kumar SHARMA, Muzaffarnagar, UP (IN)

(73) Assignee: Boston Scientific Medical Device Limited, Galway (IE)

(21) Appl. No.: 19/264,011

(22) Filed: Jul. 9, 2025

Related U.S. Application Data
(60) Provisional application No. 63/670,298, filed on Jul. 12, 2024.

(51) Int. Cl. A61M 11/02

(52) U.S. Cl. CPC A61M 11/02; A61M 2202/0007; A61M 2202/04; A61M 2205/3331

(57) ABSTRACT

A medical device may include a sheath having a proximal end, a distal end, and a lumen extending within the sheath from the proximal end to the distal end; a first distal wall disposed within the lumen of the sheath; a second distal wall disposed proximally of the first distal wall; and a seal disposed within a second space defined between the first distal wall and the second distal wall. A first space may be defined distally of the first distal wall. In a first configuration, the seal may have a proximal position relative to the second distal wall, such that the seal inhibits fluid flow around the second distal wall. In a second configuration, the seal may have a distal position relative to the second distal wall, such that the seal is configured to permit fluid flow around the second distal wall and into the first space.

### CROSS-REFERENCE TO RELATED APPLICATION(S)

This application claims the benefit of priority under 35 U.S.C. § 119 to U.S. Provisional Application No. 63/670,298, filed on Jul. 12, 2024, which is incorporated by reference herein in its entirety.

### TECHNICAL FIELD

This disclosure relates generally to medical systems, devices, and assemblies. In particular, this disclosure is directed to systems, devices, and assemblies for the delivery of one or more fluids from a distal end of a device.

### BACKGROUND

One or more fluids, or agents, may be delivered to a treatment site during medical procedures, such as endoscopic procedures. For example, during an endoscopic procedure, a user may insert a portion of a device into a body lumen of a patient. A proximal end of the device may include a handle, or gripping, portion having an actuator, for example, to control (e.g., deflect and/or position) a distal end of the device and/or to control the delivery of fluid(s) from the distal end of the device during the procedure. The proximal end of the device may also include one or more ports in fluid communication with one or more lumens of the device. In aspects, fluid(s) may be delivered through the lumen(s) (e.g., via the one or more ports) to provide treatment at the treatment site near a distal end of the device. The treatment site may be internal to the patient, and thus may be remote from the user.

### SUMMARY

Aspects of the disclosure relate to, among other things, systems, devices, and assemblies configured to deliver one or more fluids (e.g., agents). Each of the aspects disclosed herein may include one or more of the features described in connection with any of the other disclosed aspects.

According to certain aspects of the disclosure, a medical device includes a sheath having a proximal end, a distal end, and a lumen extending within the sheath from the proximal end to the distal end; a first distal wall disposed within the lumen of the sheath; a second distal wall disposed proximally of the first distal wall; and a seal disposed within a second space defined between the first distal wall and the second distal wall. A space may be defined distally of the first distal wall. In a first configuration, the seal may have a proximal position relative to the second distal wall, such that the seal inhibits fluid flow around the second distal wall. In a second configuration, the seal may have a distal position relative to the second distal wall, such that the seal is configured to permit fluid flow around the second distal wall and into the first space.

The medical device may include one or more of the following features in any combination. A control member may extend within the lumen of the sheath. The control member may be fixedly coupled to the first distal wall and may extend distally relative to the first distal wall. A diameter of a distal portion of the control member may be greater than a diameter of a proximal portion of the control member. The distal portion of the control member may be distal of the first distal wall.

The lumen of the sheath may be a first lumen. The control member may include a second lumen extending within the control member from a proximal end to a distal end of the control member. The control member may be configured to deliver a gas via the second lumen. Upon delivery of a fluid from the first lumen into the first space and a gas from the second lumen into the first space, the gas may at least partially atomize the fluid in the first space.

The first distal wall may include one or more openings configured to permit fluid flow distally past the first distal wall. A gap may be defined between a perimeter of the second distal wall and a wall defining the lumen of the sheath. In the second configuration, fluid may be permitted to flow around the second distal wall via the gap. One or more spacers may be disposed between the first distal wall and the second distal wall. The one or more spacers may be configured to define a fixed distance between the first distal wall and the second distal wall. The one or more spacers may be disposed within a central opening of the seal.

The seal may be an O-ring. The medical device may further include a biasing member disposed proximally of the second distal wall. In the first configuration, the biasing member may be in an expanded configuration. In the second configuration, the biasing member may be in a contracted configuration.

The medical device my further include a third distal wall fixed to a proximal end of the biasing member and a third space defined between the second distal wall and the third distal wall. The biasing member may be disposed within the third space.

The medical device may further include a fourth distal wall disposed proximally of the third distal wall. A plurality of distal openings may extend through the fourth distal wall. In the first configuration, a fourth space may be defined between the third distal wall and the fourth distal wall. Fluid may be permitted to flow distally (i) into the fourth space via the plurality of distal openings, (ii) around the third distal wall, and (iii) into the second space. The fourth distal wall may be sealed with respect to a wall defining the lumen of the sheath.

The medical device may further include a fifth distal wall having a plurality of proximal openings extending through the fifth distal wall, and a plurality of tubular members extending between the fourth distal wall and the fifth distal wall.

In another example, a medical device includes a handle having a stationary body and a movable body, a sheath, a first distal wall, a second distal wall, a seal, and a control member. The movable body of the handle may be movable relative to the stationary body. The sheath may be fixed to a distal end of the handle. The sheath may define a first lumen extending from a proximal end to a distal end of the sheath. The first distal wall may be disposed within the first lumen. A first space may be defined distally of the first distal wall. The second distal wall may be disposed proximally of the first distal wall. The seal may be disposed between the first distal wall and the second distal wall. The control member may define a second lumen extending from a proximal end to a distal end the control member. A proximal end of the control member may be fixed to the movable body, and a distal end of the control member may be fixed to the first distal wall. In a first position of the movable body relative to the stationary body, the seal may be configured to inhibit fluid flow around the second distal wall. In a second position of the movable body relative to the stationary body, the seal may be configured to permit fluid flow around the second distal wall and into the first space.

The medical device may include one or more of the following features in any combination. The control member may be configured to deliver a gas via the second lumen. In the second position of the movable body, and upon delivery of a gas from the second lumen into the first space, the gas may at least partially atomize a fluid within the first space. A gap may be defined between a perimeter of the second distal wall and a wall defining the first lumen of the sheath. In the second position of the movable body relative to the stationary body, fluid may be permitted to flow around the second distal wall via the gap.

In another example, a medical device includes a sheath, a wall, a plate, and a biasing member. The sheath may have a proximal end, a distal end, and a lumen extending within the sheath from the proximal end to the distal end. The wall may be disposed within the lumen of the sheath and have a plurality of openings extending therethrough. The plate may be disposed within the lumen of the sheath distally of the wall. The biasing member may be coupled to the plate. In a first configuration of the medical device, the biasing member may exert a distal force on the plate to permit fluid to flow distally from the plurality of openings and around the plate. In a second configuration of the medical device, the biasing member may exert a proximal force on the plate to inhibit fluid from flowing distally from the plurality of openings.

The medical device may include the following feature. In the second configuration, the plate may abut a distal face of the wall.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by various aspects of the elements and combinations particularly pointed out in the appended claims

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 depicts a perspective view of a medical system, according to aspects of this disclosure.

FIG. 2A depicts a perspective view of a medical device, according to aspects of this disclosure.

FIG. 2B depicts a partial cross-sectional view of a proximal portion of the medical device, according to aspects of this disclosure.

FIGS. 2C and 2D depict perspective views of a distal portion of the medical device shown in a first configuration (FIG. 2C) and a second configuration (FIG. 2B), according to aspects of this disclosure.

### DETAILED DESCRIPTION

Aspects of the disclosure include devices and methods for the delivery of fluids, such as agents. Aspects include at least partial atomization of an agent, including multi-part agents, and delivery of the at least partially atomized agent to a treatment site within a subject (e.g., patient). Although an agent is referred to herein, any other fluid or other aqueous material may be atomized and delivered using the systems, devices, and/or assemblies described herein. In particular, the disclosed aspects may be useful for delivering viscous agents by, for example, atomizing the agents prior to delivery. In aspects, the agents may be used for prophylactic treatment (e.g., to prevent bleeding) or to treat wounds or otherwise perform hemostasis.

Current devices and methods for endoscopic delivery of agents, including atomized agents are limited. Application of agents to treatment sites may be challenging, for example, due to the fact that the treatment sites may not be smooth and/or difficult to reach. Additional challenges include poor, or reduced, visibility, particularly during and/or after application of the agent. In aspects, applying the agent unevenly on the treatment site may not fully protect and/or promote healing of the tissue. Application of an agent at a treatment site, for example, can protect those sites from further tissue degradation.

In some aspects of the disclosure, a medical device may include a multi-lumen sheath configured to deliver an agent (e.g., a hemostatic agent) disposed within a lumen of the sheath and deliver a pressurized fluid (e.g., a gas) within at least one different lumen of the multi-lumen sheath. The agent and gas may remain separated through at least a portion of a length of the sheath, for example, proximal of a distal portion of the medical device. At the distal portion of the medical device, the agent may be at least partially atomized by the gas, and the atomized agent may be delivered to the treatment site (e.g., via an opening in the distal tip of the sheath). Application of the atomized agent may promote healing and decrease procedural time, among other benefits.

The medical device may be movably delivered using a lumen of a scope (endoscope, bronchoscope, gastroscope, ureteroscope, duodenoscope, colonoscope, etc.), tube, sheath, or other insertion device that has been inserted into a body cavity or lumen, for example the gastrointestinal (GI) tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens and/or organs reachable via the GI tract, any other natural opening or body tract, or bodily incision. In other aspects, the medical device may be inserted through the body cavity alone (e.g., without or alongside (outside of) the scope, tube, or sheath).

Reference will now be made in detail to aspects of the disclosure, examples of which are illustrated in the accompanying drawings. Throughout various figures, the arrows “P” and “D” depict proximal and distal directions, respectively. The term “distal” refers to a portion farthest away from a user when introducing a device into a patient. By contrast, the term “proximal” refers to a portion closest to the user when placing the device into the subject. The systems and/or devices illustrated in the accompanying drawings may not be drawn to scale. Any of the devices disclosed herein may include any of the following features, additionally or alternatively, in any combination.

As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to systems, devices, and assemblies for performing various medical procedures and/or treating portions of a subject's anatomy. For example, systems, devices, and assemblies disclosed herein may be used to treat the large intestine (colon), small intestine, cecum, esophagus, any other portion of the GI tract, and/or any other suitable anatomy (collectively referred to herein as a “treatment site”). In particular, systems, devices, and assemblies disclosed herein may be used to deliver an agent or other fluid to the treatment site. Various examples described herein include single-use or disposable medical devices that are, for example, comprised of one or more biocompatible materials.

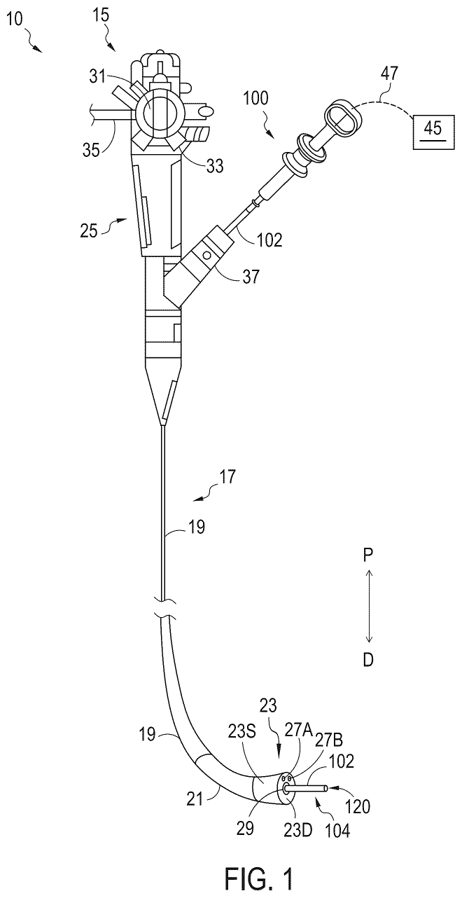

Referring to FIG. 1, a medical system 10 is shown. Medical system 10 may include a medical device 100 and a delivery device 15. Medical device 100 may be movably disposed within a lumen (e.g., a working channel) of delivery device 15. Medical device 100 may be used in conjunction with or independent of delivery device 15. Delivery device 15 may be, for example, a scope (e.g., endoscope, bronchoscope, gastroscope, ureteroscope, duodenoscope, colonoscope, etc.), tube, sheath, or insertion device configured for insertion into a body cavity or lumen of a subject.

In aspects, delivery device 15 may include an insertion portion 17 comprising a shaft 19 having an articulation portion 21 and a distal tip 23. A handle 25 may be connected at a proximal end of shaft 19. Shaft 19 may include one or more lumens extending therethrough. For example, a working channel may extend through shaft 19. In aspects, portions or an entirety of shaft 19 may be flexible, rigid, and/or semi-rigid.

Distal tip 23 may include an imaging device 27A (e.g., camera) and/or one or more lighting elements 27B (e.g., LEDs, optical fibers, etc.) disposed on a distalmost face 23D of distal tip 23. Although not shown, in some aspects, imaging device 27A and/or lighting elements 27B may, additionally or alternatively, be disposed on a side surface 23S of distal tip 23 (e.g., proximal to distalmost face 23D of distal tip 23). One or more electrical cables or wires may extend from the proximal end of delivery device 15 (e.g., handle 25), through shaft 19, and to imaging device 27A and/or lighting element(s) 27B. The electrical cables or wires may provide electrical power and/or controls to imaging device 27A, lighting element(s) 27B, and/or other electrical devices in or on distal tip 23. In aspects, the electrical cables or wires may carry imaging signals from distal tip 23 proximally, for example, to be processed by a controller and/or displayed on a display. Distal tip 23 may further include one or more openings 29 disposed on distalmost face 23D. Opening(s) 29 may additionally or alternatively be disposed on side surface 23S of distal tip 23. Opening(s) 29 may be distal openings of one or more lumens (e.g., a working channel) extending through shaft 19.

Handle 25 may include one or more mechanisms configured to articulate or otherwise move articulation portion 21 in one or more directions, thereby moving distal tip 23 in one or more directions. For example, a plurality of actuating elements, such as cables or wires, may extend distally from a proximal end of delivery device 15 (e.g., such as handle 25), through one or more lumens of shaft 19, to articulation portion 21 and/or distal tip 23. For example, the actuating elements may be directly or indirectly coupled to first and second actuating devices 31, 33, which may control articulation of articulation portion 21 in multiple directions, such as up, down, left, and/or right (e.g., respective to a longitudinal axis of shaft 19).

Devices 31, 33 of handle 25, may be, for example, rotatable knobs that rotate about their axes to push/pull actuating elements extending through delivery device 15. Additional devices (e.g., knobs, buttons, levers, etc.) may be configured to control other aspects of delivery device 15. For example, additional devices may control aspects of distal tip 23, and/or aspects of elements attached to distal tip 23, such as an end effector. In some aspects, the additional devices may control up/down movement of an elevator (not shown) of distal tip 23.

An umbilicus 35 may be fixed to, or extend from, a portion of handle 25. For example, umbilicus 35 may be configured for introducing fluid, suction, and/or wiring for electronic components to handle 25. Umbilicus 35 may include a connector for connecting umbilicus 35 to one or more of a controller, a display, etc., not shown.

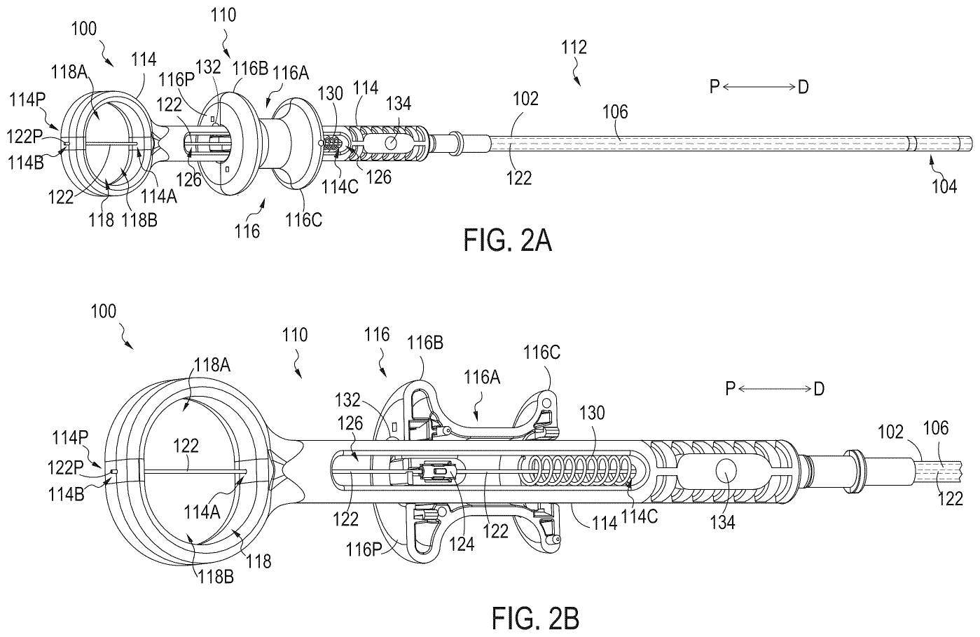

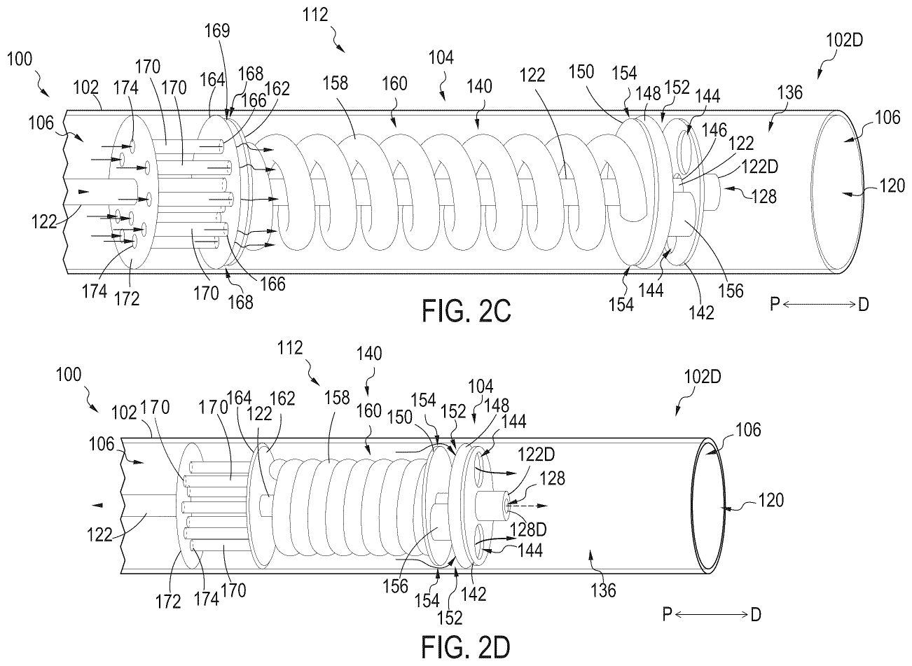

Handle 25 may further include a port 37 for introducing and/or removing tools, fluids, devices, and/or other materials to and/or from the subject. For example, port 37 may be in fluid communication with one or more lumens (e.g., the working channel) of shaft 19 and distal tip 23. Port 37 may be configured to receive a portion (e.g., a sheath 102) of medical device 100. For example, sheath 102 of medical device 100 may be inserted through an opening of port 37 and a lumen of shaft 19. A distal portion 104 of sheath 102 of medical device 100 may be extended distally from distal tip 23, for example, via opening 29. Medical device 100 may be movable (e.g., translatable proximally and/or distally) within the lumen of delivery device 15, for example, controlling a length of medical device 100 that extends distally of opening 29. Aspects of medical device 100 are discussed in more detail with regards to FIGS. 2A-2D.

Still referring to FIG. 1, a gas source 45 may be fluidly coupled to medical device 100. Gas source 45 may be fluidly connected to medical device 100 via, for example, a fluidic connector and/or a fluidic pathway 47 (shown in broken lines). Fluidic pathway 47 may be a Luer lock connection, a tube, a lumen, a port, or any other device configured to fluidly connect medical device 100 to gas source 45. Gas source 45 may be configured to deliver a pressurized gas to a distal portion 104 of sheath 102 of medical device 100, for example, via a lumen 106 (FIGS. 2A-2D) of medical device 100 (shown in more detail in the following figures). In aspects, an at least partially atomized agent may be delivered from lumen 106 (FIGS. 2A-2D) via a distalmost opening 120 of lumen 106.

Gas source 45 may be configured to supply nitrogen, oxygen, nitrous oxide, medical air, atmospheric air, and/or any other type of medical gas to medical device 100. In some aspects, gas source 45 may be a syringe, a canister, a compressor, an insufflator, or any other medical device or component configured to deliver gas or air to medical device 100. Although gas source 45 is shown as being fluidly coupled to a proximalmost end of medical device 100, gas source 45 may be fluidly coupled to medical device 100 elsewhere along a longitudinal length of medical device 100. In some aspects, gas source 45 may be fluidly coupled to delivery device 15, as well.

FIG. 2A illustrates a perspective view of medical device 100 having a handle portion 110 and an insertion portion 112 including a sheath 102. FIG. 2B illustrates a partial cross-sectional view of handle portion 110 of medical device 100. One or more portions of handle portion 110 may be movable or manipulatable to control various aspects of distal portion 104 of sheath 102, shown in FIGS. 2B and 2C. Described in further detail with respect to FIGS. 2C and 2D, one or more portions of distal portion 104 of sheath 102 may be movable to facilitate, for example, delivery of an atomized agent from medical device 100.

Referring to FIGS. 2A and 2B, handle portion 110 of medical device 100 may include a stationary body 114 and a movable body 116 (e.g., an actuator). A proximal portion of stationary body 114 may include a finger ring 118, for example, to receive a user's thumb. Sheath 102 may be coupled (e.g., directly or indirectly) or otherwise connected to a distal end of stationary body 114 via, for example, an adhesive, a mechanical fit, a snap fit, a press fit, a weld (e.g., a laser weld), or any other coupling techniques commonly known in the art.

Movement of movable body 116 relative to stationary body 114 may control movement (e.g., extension or retraction) of a portion of distal portion 104 of sheath 102, discussed in further detail below. In some aspects, movement of movable body 116 relative to stationary body 114 may control the delivery of an at least partially atomized agent from distalmost opening 120 of sheath 102. To control the movement of aspects of distal portion 104, a control element 122 may be coupled to movable body 116 and to one or more components of a fluid delivery assembly disposed within distal portion 104 (shown in more detail in FIGS. 2C and 2D).

Control element 122 may be comprised of any flexible or semi-flexible material, such as, for example, metals, polymers, metal-polymer composites, and the like. Control element 122 may be in the form of a tube, a sheath, etc. For example, control element 122 may include at least one lumen 128 (see FIGS. 2C and 2D) extending longitudinally therethrough (e.g., from proximalmost end 122P to distalmost end 112D of control element 122, shown in FIGS. 2C and 2D).

Referring to FIGS. 2A and 2B, control element 122 may be fixed within movably body via a fixing member 124. Fixing member 124 may be a crimp, a ferrule, a bead of adhesive, a weld, or any other component or technique configured to secure control element 122 within movable body 116. In these aspects, fixing member 124 may be fixed directly or indirectly to control element 122 and/or to movable body 116.

Control element 122 may extend proximally from a proximal face 116P of movable body 116. For example, control element 122 may extend from proximal face 116P through a first channel 114A of stationary body 114. First channel 114A may be proximal of movable body 116. In aspects, a proximal opening of first channel 114A may be disposed on a wall defining finger ring 118. A distal opening of first channel 114A may be disposed on a wall defining a proximal end of a slot 126 of stationary body 114. In these aspects, control element 122 may extend proximally from movable body 116, through the distal opening of first channel 114A, through first channel 114A, and out of the proximal opening of first channel 114A.

In some aspects, control element 122 may be configured for coupling to gas source 45 (e.g., via fluidic pathway 47) (see FIG. 1) at or near the proximal opening of first channel 114A. In other aspects, control element 122 may continue to extend proximally through the opening of finger ring 118. For example, control element 122 may divide finger ring 118 into two openings. For example, control element 122 may divide finger ring 118 such that a first opening 118A and a second opening 118B are defined on either side of control element 122.

Control element 122 may continue to extend proximally, for example, through a second channel 114B. Second channel 114B may be proximal of first channel 114A. In aspects, a distal opening of second channel 114B may be disposed on a proximal wall of finger ring 118. A proximal opening of second channel 114B may be disposed on a proximalmost face 114P of stationary body 114. Control element 122 may extend proximally through second channel 114B. In aspects, control element 122 may be configured to couple to gas source 45 (e.g., via fluidic pathway 47) (see FIG. 1) at or near the proximal opening of second channel 114B.

In aspects, control element 122 may continue proximally relative to proximalmost face 114P of stationary body 114 such that, for example, a proximalmost end 122P is proximal of proximalmost face 114P. Proximalmost end 122P of control element 122 may be configured to fluidly couple to gas source 45 (FIG. 1). Control element 122 may be movably disposed within first channel 114A and second channel 114B. For example, when movable body 116 is in a proximal position, control element 122 may extend more proximally from proximalmost face 114P as compared to when movable body 116 is in a distal position. In alternatives, a fixed tubular body may extend from proximalmost face 114P to movable body, such that a lumen of the fixed tubular body is in fluid communication with lumen 128 of control element 122.

Control element 122 may extend distally through stationary body 114 via a third channel 114C. For example, third channel 114C may be distal relative to first channel 114A and second channel 114B. Third channel 114C may be distal relative to movable body 116. Control element 122 may continue to extend distally through lumen 106 of sheath 102. Control element 122 is shown as extending through lumen 106 of sheath 102 using broken lines in FIGS. 2A and 2B. Control element 122 may be longitudinally movable within third channel 114C of stationary body 114 and lumen 106 of sheath 102. Control element 122 may also be longitudinally movable within first channel 114A and second channel 114B. Accordingly, when movable body 116 is moved distally and/or proximally relative to stationary body 114, control element 122 may translate distally and/or proximally within third channel 114C and lumen 106.

Referring still to FIGS. 2A and 2B, a proximal biasing member 130 may be at least partially disposed between stationary body 114 and movable body 116. For example, proximal biasing member 130 may be at least partially disposed within slot 126 defined within stationary body 114. Proximal biasing member 130 may bias movable body 116 in a distal or proximal position relative to stationary body 114. For example, proximal biasing member 130 may be a coiled spring, a wave spring, extension spring, etc. that biases movable body 116 in a proximal position. Alternatively, proximal biasing member 130 may be a compression spring that biases movable body 116 in a distal position. Although springs are discussed herein, proximal biasing member 130 may be any device or component configured to bias a position of movable body 116 relative to stationary body 114.

Movable body 116 may include one or more reduced diameter sections or grooves 116A, for example, between a proximal ridge 116B and a distal ridge 116C, which may help to facilitate the user's grip of movable body 116. For example, the user may place movable body 116 between two fingers such that the user's fingers are positioned within groove(s) 116A. Accordingly, movement of the user's fingers (i.e., relative to the user's thumb in finger ring 118) controls the position of movable body 116 relative to stationary body 114, and thus the movement of control element 122 and/or one or more portion(s) of distal portion 104 of sheath 102.

Stationary body 114 and/or movable body 116 may each have one or more features 132 (e.g., a protrusion, an indentation, a mark, etc.) to provide tactile or visual feedback to the user of the relative position of movable body 116 relative to stationary body 114. Thus, feature(s) 132 may provide visual or tactile feedback of the relative position or configuration of distal portion 104. For example, as movable body 116 is advanced distally relative to stationary body 114, thus actuating one or more portion(s) of distal portion 104 of sheath 102, movable body 116 may have to overcome, for example, feature(s) 132.

Feature(s) 132 may signal (e.g., via a tactile signal or feedback, via a visual signal, etc.) to the user that distal portion 104 of sheath 102 has been actuated, or is in an extended position. Feature(s) 132 may be a bump, a cantilevered protrusion, an indentation, a ridge, or any combination of the like. For example, feature(s) 132 on one side of handle portion 110 may be a bump and feature(s) 132 on an opposing side of handle portion 110 may be a cantilevered protrusion. Many other combinations are also contemplated. Additionally or alternatively, the user may utilize one or more markings on handle portion 110 (e.g., on stationary body 114 and/or movable body 116) or other visual indicators to determine a status, position, actuation status, etc. of one or more portion(s) of distal portion 104 of sheath 102.

In some aspects, stationary body 114 may include a port 134. Port 134 may be in fluid communication with lumen 106 of sheath 102. Port 134 may be configured to permit the delivery of a fluid or agent into lumen 106 of sheath 102 and to distal portion 104. In other aspects, the fluid or agent may be pre-loaded into lumen 106 of sheath 102, for example, prior to the insertion of sheath 102 into the subject.

In aspects, sheath 102 may be removably coupled to handle portion 110. Lumen 106 of sheath 102 may be pre-filled or pre-loaded with an agent. In these aspects, multiple sheaths 102 may be used during a single procedure, for example, by attaching and detaching multiple sheaths 102 to handle portion 110.

FIGS. 2C and 2D illustrate distal portion 104 of sheath 102 of medical device 100 in a first configuration (FIG. 2C) and in a second configuration (FIG. 2D). As previously discussed with reference to FIGS. 2A and 2B, sheath 102 may include a lumen 106 extending therethrough. A distalmost opening 120 of lumen 106 may be disposed on a distalmost end 102D of sheath 102. Accordingly, a direction of fluid flow may be in a direction that is parallel to a longitudinal axis of sheath 102. Additionally or alternatively, distalmost opening 120 may be disposed on a side surface of sheath 102 (e.g., proximal of distalmost end 102D of sheath 102). Accordingly, a direction of fluid flow may be in a direction that is transverse to the longitudinal axis of sheath 102. Unless otherwise specified, fluid flow is illustrated in FIGS. 2C and 2D using solid lines and arrows extending in the distal direction.

As previously discussed with reference to FIGS. 2A and 2B, control element 122 extends longitudinally through lumen 106. In aspects, control element 122 may be parallel to or coaxial with the central longitudinal axis of sheath 102. A distalmost end 122D of control element 122 may terminate proximally of distalmost end 102D of sheath 102 in both the first configuration and the second configuration. In these aspects, a first space 136 may be defined distally relative to distalmost end 122D of control element 122 and proximally of distalmost opening 120.

A diameter of distalmost end 122D of control element 122 may be greater than a diameter of a portion of control element 122 that is proximal of distalmost end 122D. In some aspects, distalmost end 122D of control element 122 may include a crimp, a ferrule, a bead of glue, a weld, or any other element or technique that results in the distalmost end 122D of control element 122 having a greater diameter than a portion of control element 122 that is proximal of distalmost end 122D. Shown in FIG. 2D, lumen 128 of control element 122 may extend through distalmost end 122D such that a distalmost opening 128D of lumen 128 is disposed on a distalmost face of distalmost end 122D.

A fluid delivery assembly 140 may be disposed within distal portion 104 of sheath 102, for example, within lumen 106. As will be described in further detail below, fluid delivery assembly 140 may be comprised of a series of spaces divided by a series of walls. For example, fluid delivery assembly 140 may include first space 136 defined distally relative to distalmost end 122D of control element 122 and proximally of distalmost opening 120. A proximal end of first space 136 may be at least partially defined by a first distal wall 142. A second space 152 may be defined between first distal wall 142 and a second distal wall 150. Second space 152 may be proximal of first space 136. A third space 160 may be defined between second distal wall 150 and a third distal wall 164. Third space 160 may be proximal of second space 152. Lumen 106 may continue proximally of fourth distal wall 172. Fluid may be disposed within lumen 106, for example, proximally of fourth distal wall 172.

Explained in further detail below, fluid may be configured to flow proximally to distally within fluid delivery assembly 140. Depending on the configuration of fluid delivery assembly 140 (e.g., the first configuration (FIG. 2C) or the second configuration (FIG. 2D)), fluid may be permitted to flow in/through certain walls and in/through certain spaces. For example, in the first configuration, shown in FIG. 2C, fluid disposed proximally of fourth distal wall 172 within lumen 106 may flow distally through a plurality of proximal openings 174 extending through fourth distal wall 172 and third distal wall 164. The fluid flow is illustrated in FIG. 2C using solid arrows extending in a distal direction. The fluid may flow into third space 160. In aspects, the fluid may remain disposed within third space 160, for example, until fluid delivery assembly 140 is transitioned into the second configuration (FIG. 2D).

In the second configuration, the fluid may flow from third space 160, distally past second distal wall 150, and into second space 152. The fluid may continue to flow through openings that extend through first distal wall 142 and into first space 136. The fluid may then flow distally out of distalmost opening 120, and, for example, be delivered to the target site. Described in more detail below, control element 122 may be configured to transition fluid deliver assembly 140 between the first and second configurations. Aspects of fluid delivery assembly 140 are discussed in further detail below.

Referring still to FIGS. 2C and 2D, first distal wall 142 (e.g., plate) may be disposed proximally of distalmost end 122D of control element 122. A diameter of first distal wall 142 may be equal to or less than an inner diameter of lumen 106 of sheath 102. One or more openings 144 may extend through first distal wall 142 (e.g., through an entire thickness of first distal wall 142). Opening(s) 144 may be configured to permit fluid flow through first distal wall 142 in at least some configurations of fluid delivery assembly 140.

As shown in FIG. 2C, control element 122 may extend through a central opening 146 of first distal wall 142 such that distalmost end 122D is disposed distally of first distal wall 142. Opening(s) 144 may be disposed around central opening 146, for example, on first distal wall 142. Distalmost end 122D of control element 122 may be configured as a stop to prevent or prohibit first distal wall 142 from moving distally (e.g., past distalmost end 122D). First distal wall 142 may be fixed to distalmost end 122D. In other aspects, first distal wall 142 may be independent of (e.g., not fixed to) distalmost end 122D.

A seal 148 may be disposed proximally of first distal wall 142, for example, between first distal wall 142 and second distal wall 150. Seal 148 may be disposed within second space 152 defined between first distal wall 142 and second distal wall 150. Seal 148 may be a compressible member such as, for example, an O-ring or similar structure. An outer diameter of seal 148 may be at least equal to the inner diameter of lumen 106. Seal 148 may be torus-, or ring-shaped such that an opening (not shown) is centrally defined. An interference between seal 148 and an inner surface of lumen 106 may inhibit fluid from flowing distally past seal 148, for example, when fluid delivery assembly 140 is in the first configuration (FIG. 2C).

A central opening (obscured from view in FIGS. 2C and 2D) may be disposed on second distal wall 150. The central opening may be configured to permit control element 122 to extend through second distal wall 150. In aspects, the central opening may be sealed against control element 122, for example, to prevent fluid from flowing distally or proximally relative to second distal wall 150 via the central opening. In some aspects, second distal wall 150 may be fixed to control element 122.

A diameter of second distal wall 150 may be less than the inner diameter of lumen 106. In these aspects, a gap 154 may be defined around second distal wall 150, for example, between the internal wall of sheath 102 and an outer perimeter of second distal wall 150. One or more spacers 156 may extend between first distal 142 and second distal wall 150. For example, spacer(s) 156 may be configured to maintain a position, or a distance, of first distal wall 142 relative to second distal wall 150 such that, for example, a volume of second space 152, discussed below, is maintained. In some aspects, seal 148 may be movable within second space 152. For example, seal 148 may move within second space 152 as a result of being pushed and/or pulled by first distal wall 142 and/or second distal wall 150 (e.g., when control element 122 is pushed distally or pulled proximally).

With seal 148 in a proximal position, as shown in FIG. 2C, seal 148 may prevent fluid flow distally into second space 152 from third space 160. Seal 148 may block gap 154 surrounding second distal wall 150, thereby seal 148 preventing, inhibiting, or prohibiting fluid from flowing distally past gap 154 surrounding second distal wall 150. In these aspects, fluid may be prevented from flowing into second space 152. Accordingly, in the first configuration (e.g., when seal 148 is in the proximal position), fluid flow may be prevented or prohibited. For example, seal 148 may be configured block, or seal, gap 154.

With seal 148 in a distal position, as shown in FIG. 2D, fluid may flow distally into second space 152 from third space 160 via gap 154. In the second configuration, the fluid may continue to flow from second space 152 into first space 136, for example, via opening(s) 144 of first distal wall 142. In the second configuration (e.g., when seal 148 is in the distal position), distal fluid flow into first space 136 may be permitted.

In aspects, first space 136 may provide a space for the fluid and the gas from control element 122 to at least partially mix or combine. In examples, the fluid may be delivered from a proximal portion of sheath 102 (e.g., from third space 160). For example, with fluid delivery assembly 140 in the second configuration, the fluid may flow past second distal wall 150 (e.g., via gap 154), through second space 152, and through opening(s) 144 of first distal wall 142. The gas may be delivered via a distal opening of lumen 128 of control element 122. As the gas flows from lumen 128 of control element 122 and into first space 136, the fluid within first space 136 may become at least partially atomized and/or at least partially aerosolized. The fluid, including the at least partially atomized and/or aerosolized fluid, may be delivered from distal portion 104, through distalmost opening 120. The at least partially atomized fluid may then be applied to target tissue, for example, at the treatment site.

To transition fluid delivery assembly 140 between the first, proximal position (FIG. 2C) and the second, distal position (FIG. 2D), control element 122 may be pulled or translated proximally and distally within lumen 106 of sheath 102. For example, as control element 122 is initially pulled or translated proximally within lumen 106, first distal wall 142 and second distal wall 150 may initially simultaneously be translated proximally while seal 148 remains stationary. Proximal movement of control element 122 is illustrated using a broken arrow extending proximally in FIG. 2D. In some aspects, as control element 122 is pulled or translated proximally, seal 148 may abut a proximal face of first distal wall 142. As control element 122 is further pulled or translated proximally, seal 148 may remain abutted against first distal wall 142, and may be translated proximally within lumen 106 of sheath 102. In other words, a distance between seal 148 and distalmost opening 120 may remain the same or increase, for example, when fluid deliver assembly 140 is transitioned into the second configuration. With seal 148 abutted against first distal wall 142 (e.g., in the second configuration), seal 148 may be in a distal position within second space 152 and fluid flow may be permitted around second distal wall 150 (e.g., via gap 154).

When control element 122 is pushed or translated distally, first distal wall 142 and second distal wall 150 simultaneously may move distally relative to seal 148. Distal movement of control element 122 is illustrated using a broken arrow extending in the distal direction in FIG. 2C. Accordingly, seal 148 may be in a proximal position within second space 152 such that seal 148 abuts against a distal face of second distal wall 150. Fluid flow may be prevented or prohibited from flowing around second distal wall 150 with seal in the proximal position. As control element 122 is pushed or translated further in the distal direction, seal 148 may remain abutted against second distal wall 150, but may be translated distally within lumen 106 of sheath 102. In other words, a distance between seal 148 and distalmost opening 120 may remain the same or decrease, for example, when fluid deliver assembly 140 is transitioned into the first configuration.

Accordingly, in some aspects, seal 148 may remain stationary within lumen 106. For example, control element 122, first distal wall 142, and second distal wall 150 may move relative to seal 148. In these aspects, seal 148 may be integrally formed with and/or fixed to a wall defining sheath 102. In other aspects, seal 148 may be movable within lumen 106. For example, seal 148 may be permitted to translate proximally and/or distally within 106 as a result of being pushed or pulled by first distal wall 142 or second distal wall 150.

As discussed above, control element 122 may be translated proximally and/or distally via movable body 116 of FIGS. 2A and 2B. For example, referring back to FIGS. 2A and 2B, when movable body 116 is in a distal position relative to stationary body 114, fluid delivery assembly 140 may be in the first configuration (FIG. 2C). When movable body 116 is in a proximal position relative to stationary body 114, fluid delivery assembly 140 may be in the second configuration (FIG. 2D).

Fluid delivery assembly 140 may further include a distal biasing member 158 (e.g., a spring) disposed proximally of second distal wall 150. Biasing member 158 may be configured to maintain a position of second distal wall 150 relative to third distal wall 164 in the first and second configurations. For example, in the first configuration (FIG. 2C), biasing member 158 may have a natural length (e.g., biasing member 158 may be in a neutral state) or a greater length than the natural length of biasing member 158 (e.g., biasing member 158 may be expanded). Thus, in the first configuration, biasing member 158 may exert a distal force on a sealing plate 162 that is fixedly coupled to biasing member 158. In some aspects, for example, when fluid delivery assembly 140 is in the first configuration (FIG. 2C), biasing member 158 may be configured to enable fluid flow into third space 160 from a portion of lumen 106 that is proximal of fourth distal wall 172, for example, by translating/pulling sealing plate 162 (fixed to a proximal end of biasing member 158) distally relative to third distal wall 164. Fluid flow around sealing plate 162 is illustrated in FIG. 2C using curved arrows extending in a distal direction, for example.

In some examples, in the second configuration of FIG. 2D, biasing member 158 may have a length that is shorter than a natural length of biasing member 158. In the second configuration, biasing member 158 may thus exert a proximal force on sealing plate 162. Accordingly, biasing member 158 may be configured to prevent or inhibit fluid flow into third space 160 from a portion of lumen 106 that is proximal of fourth distal wall 172, for example, by translating sealing plate 162 proximally against third distal wall 164.

Biasing member 151 may be disposed within third space 160 defined between second distal wall 150 and sealing plate 162. A proximal end of distal biasing member 158 may be fixed to sealing plate 162 and/or a distal end of distal biasing member 158 may be fixed to second distal wall 150. Control element 122 may extend proximally and distally through distal biasing member 158.

A diameter of sealing plate 162 may be less than the inner diameter of lumen 106. In some aspects, sealing plate 162 may be movable relative to a third distal wall 164 that is proximal of sealing plate 162. Third distal wall 164 may have a diameter that is at least equal to a diameter of lumen 106. In aspects, third distal wall 164 may be stationary with respect to sheath 102. For example, third distal wall 164 may be fixed to sheath 102. Third distal wall 164 may include a plurality of distal openings 166 extending therethrough. Control element 122 may extend through a central opening of each of sealing plate 162 and third distal wall 164. Control element 122 may be movable relative to sealing plate 162 and third distal wall 164.

Referring still to FIGS. 2C and 2D, fluid delivery assembly 140 may further include a plurality of separators 170. The plurality of separators 170 may be tubular members extending between third distal wall 164 and fourth distal wall 172. The separators may extend through plurality of distal openings 166 or may abut proximal ends of distal openings 166. The plurality of distal openings 166 extending through third distal wall 164 may be distal openings of the plurality of separators 170. Fluid may flow from lumen 106 (e.g., proximally of fourth distal wall 172) into lumens of separators 170. Plurality of separators 170 may be configured to separate the fluid flowing distally within lumen 106 (e.g., from a portion of lumen 106 that is proximal of fourth distal wall 172) and into fluid delivery assembly 140. In aspects, separation of the fluid within the plurality of separators 170 may assist in atomizing the fluid (e.g., once the fluid has been delivered into first space 136).

A proximal end of each of the plurality of separators 170 may be fixed to fourth distal wall 172. Plurality of proximal openings 174 may extend through an entirety of fourth distal wall 172. A diameter of fourth distal wall 172 may be equal to the inner diameter of lumen 106, and fourth distal wall 172 may be sealed with respect to an inner surface of sheath 102. Control element 122 may extend through fourth distal wall 172, for example, via a central opening (not shown). Control element 122 may be movable relative to fourth distal wall 172.

In the first configuration (FIG. 2C), fourth space 169 may be defined between sealing plate 162 and third distal wall 164. Thus, fluid disposed within lumen 106 proximally of fourth distal wall 172 may be permitted to flow distally from lumen 106 (e.g., proximal of fourth distal wall 172), through the separators 170, out of the plurality of distal openings 166. The fluid may be permitted to continue to flow around sealing plate 162 and into third space 160, for example, via a gap 168 surrounding a perimeter of sealing plate 162. In these aspects, fluid may only be permitted to flow distally through the plurality of separators 170 via the plurality of proximal openings 174 and the plurality of distal openings 166 (illustrated using solid arrows extending in the distal direction in FIG. 2C). In other words, fluid may not travel through the space around separators 170. The fluid may be contained in third space 160, for example, until fluid delivery assembly 140 is transitioned into the second configuration (FIG. 2D).

In the second configuration (FIG. 2D), sealing plate 162 may be configured to abut, or press against, third distal wall 164. In these aspects, fourth space 169 may be reduced or eliminated. For example, in the second configuration, sealing plate 162 may be configured to close, or seal the plurality of distal openings 166 disposed on the distal face of third distal wall 164. Fluid may thereby be prevented from flowing into third space 160 from the plurality of distal openings 166 in the second configuration. Furthermore, in the second configuration, distal biasing member 158 may be in a compressed, or contracted configuration, thereby decreasing a volume of third space 160. As the volume of third space 160 decreases, the fluid may be urged to flow distally, for example, around second distal wall 150, as discussed above.

In the first configuration, the fluid may be urged to flow into third space 160, for example, due to a negative pressure that may be created by transitioning fluid delivery assembly 140 between the first and second configurations. For example, as distal biasing member 158 is extended, or expanded, (e.g., when fluid delivery assembly 140 is transitioned from the second configuration to the first configuration), a negative pressure may be created within third space 160 such that the fluid contained within lumen 106 proximally of fourth distal wall 172 is urged to flow distally into third space 160. The negative pressure may be created within third space 160, for example, due to a volume of third space 160 increasing. The negative pressure may also urge the fluid to flow through the plurality of separators 170 and around sealing plate 162 (e.g., via gap 168).

To transition fluid delivery assembly 140 into the second configuration (FIG. 2D), control element 122 may be translated proximally (e.g., by moving movable body 116 of FIGS. 2A and 2B proximally relative to stationary body 114). Proximal movement of control element 122 is illustrated in FIG. 2D using a broken arrow extending in the proximal direction. As control element 122 is translated proximally, first distal wall 142 and second distal wall 150 may be translated proximally as third distal wall 164 and fourth distal wall 172 remain stationary. Seal 148 may also be abutted against first distal wall, thereby creating gap 154 around second distal wall 150. Accordingly, the volume of third space 160 may decrease, thereby urging the fluid from third space 160 distally and around second distal wall 150 (e.g., via gap 154). The fluid may continue to flow through second space 152, through the central opening of seal 148, and through opening(s) 144 of first distal wall 142. Fluid flow through gap 154 and through opening(s) 144 is illustrated using solid curved arrows extending in the distal direction.

In the first configuration, for example, when the fluid is distal of first distal wall 142, the fluid may come in contact with the gas delivered via lumen 128 of control element 122. The delivery of gas is illustrated in FIG. 2D using a broken arrow extending in the distal direction. In these aspects, the fluid may become at least partially atomized and delivered out of distalmost opening 120 of lumen 106 and to the target treatment site. Although not shown in FIG. 2C, gas may also be delivered in the first configuration.

The fluid delivery assembly 140 may transition between the first and second configurations one or more times, for example, throughout a procedure. In some aspects, fluid delivery assembly 140 may be configured to deliver discrete doses of the atomized fluid to the target tissue. In other aspects, medical device 100 may be configured to deliver a fluid (e.g., a non-atomized fluid). In further aspects, medical device 100 may be configured to deliver a first fluid via fluid delivery assembly 140 and a second fluid via control element 122. In aspects, the first and second fluid may react with each other prior to being delivered from sheath 102 (e.g., within first space 136), and/or upon being delivered to the target site.

During use, distal portion 104 of sheath 102 of medical device 100 may be navigated to a target site. Handle portion 110 of medical device 100 may remain outside of the subject. In some aspects, a fluid may be disposed within lumen 106 of sheath 102, for example, proximal of fluid delivery assembly 140 disposed within distal portion 104. For example, the fluid may be pre-loaded within lumen 106 (e.g., prior to sheath 102 being inserted into the subject) and/or the fluid may be injected into lumen 106 (e.g., via port 134 disposed on handle portion 110).

Medical device 100 (e.g., fluid delivery assembly) may be in the first configuration (FIG. 2C) or the second configuration (FIG. 2D) upon navigating and/or reaching the target site. Assuming medical device 100 is in the first configuration (FIG. 2C), movable body 116 of handle portion 110 may be in a distal position relative to stationary body 114.

As discussed above, in the first configuration (FIG. 2C), fluid disposed within lumen 106 (e.g., proximal of fluid delivery assembly 140) may be permitted to flow distally within a portion of fluid delivery assembly 140. For example, the fluid may be permitted to flow distally into plurality of separators 170 (e.g., via plurality of proximal openings 174 of fourth distal wall 172 and plurality of distal openings 166 of third distal wall 164). In the first configuration, sealing plate 162 may be spaced from third distal wall 164, thereby permitting fluid flow into third space 160. Sealing plate 162 may be spaced from third distal wall 164 as biasing member 158 is in an expanded configuration. Biasing member 158 may be in an expanded configuration, for example, due to control element 122 being pushed distally within lumen 106.

In the first configuration, fluid flow may be prevented from flowing distally past third space 160. For example, in the first configuration, seal 148 may be abutted against second distal wall 150 so as to close gap 154 that extends circumferentially around second distal wall 150. Accordingly, fluid may be contained within third space 160 when fluid delivery assembly 140 is in the first configuration.

Transitioning fluid delivery assembly 140 from the first configuration (FIG. 2C) to the second configuration (FIG. 2D) permits fluid delivery from third space 160 to second space 152, and to first space 136. Accordingly, the fluid may be delivered to the target site in the second configuration. Movable body 116 may be moved to a proximal position relative to stationary body 114. Accordingly, control element 122 may be translated proximally within lumen 106. As control member is translated proximally, first distal wall 142 and second distal wall 150 may simultaneously move proximally within lumen 106. Accordingly, seal 148 may abut first distal wall 142 in the second configuration, as discussed above.

With seal 148 abutting first distal wall 142, gap 154 may open, thereby permitting fluid to flow from third space 160, around second distal wall 150 and into second space 152. Fluid may continue to flow distally, for example, through the central opening of seal 148 and through opening(s) 144 of first distal wall 142. The fluid may then at least partially interact with gas delivered via control element 122 and, in some aspects, become at least partially atomized. The at least partially atomized fluid may be delivered to the target site.

In some aspects, fluid disposed within third space 160 may be forced distally, for example, as the volume of third space 160 decreases. The volume of third space 160 may decrease as control element 122 is pulled proximally and third distal wall 164 remains stationary within lumen 106. Furthermore, in the second configuration, as control element 122 is pulled proximally, biasing member 158 may be compressed. As biasing member 158 is compressed, sealing plate 162 may abut third distal wall 164, thereby closing, or sealing, plurality of distal openings 166. Accordingly, fluid flow into third space 160 may be prevented.

Fluid delivery assembly 140 may be transitioned back into the first configuration (FIG. 2C), for example, by translating control element 122 distally. As control element 122 is translated distally, first distal wall 142 and second distal wall 150 are translated distally such that seal 148 abuts second distal wall 150 and closes gap 154.

Furthermore, as fluid delivery assembly 140 is transitioned back into the first configuration, biasing member 158 may extend and sealing plate 162 may be separated from third distal wall 164, thereby permitting fluid flow into third space 160. In some aspects, for example, as the volume of third space 160 increases, a negative pressure may be created within third space 160. Accordingly, fluid may be pulled distally from lumen 106 (e.g., proximal of fourth distal wall 172), through the plurality of separators 170 (e.g., via plurality of proximal openings 174 of fourth distal wall 172 and plurality of distal openings 166 of third distal wall 164), and into third space 160.

Fluid delivery assembly 140 may transition between the first and second configurations any number of times throughout a procedure. After a desired amount of fluid is delivered to the target site, medical device 100 may be removed from the subject.

Each of the aforementioned systems, devices, and assemblies may be used to protect and/or treat treatment sites by delivering an atomized agent(s) (e.g., a gel, a powder, or one or more other prophylactic agents) to the treatment site. By providing a medical device configured to deliver an agent in atomized form, known problems associated with uneven application of the agent to the treatment site and/or other aspects of invasive surgical procedures may be reduced or avoided. For example, the delivery of one or more agents in atomized form may help to form a smooth (e.g., conformal) and/or even layer of agent (e.g., adhesive) across a surface of the treatment site, which may help to protect the treatment site and/or promote healing.

Aspects of this disclosure may help to apply the agent(s) in a thin layer, which may help facilitate material (e.g., gel) properties, and/or result in a lower profile cover or layer over the treatment site, which may help to reduce the risk of the applied agent(s) becoming dislodged, being sheared, or otherwise separating from the treatment site (e.g., from other fluids and/or solids adjacent to the treatment site, for example, the GI tract). For example, aspects of this disclosure may help to form one or more layers of agent(s) to help minimize delayed bleeds, potential perforations, stricture formations, etc. Accordingly, physicians or other users may reduce the overall procedure time, increase efficiency of procedures, and/or avoid unnecessary harm to a subject's body caused by limited ability of other tools/devices to treat perforations, post-surgical leaks, and/or other wounds that might result from endoscopic and open surgical procedures of a body lumen (e.g., the GI tract).

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only, with a true scope of the disclosure being indicated by the following claims and their equivalents.

We claim:

1. A medical device, comprising:
a sheath having a proximal end, a distal end, and a lumen extending within the sheath from the proximal end to the distal end;
a first distal wall disposed within the lumen of the sheath, wherein a first space is defined distally of the first distal wall;
a second distal wall disposed proximally of the first distal wall; and
a seal disposed within a second space defined between the first distal wall and the second distal wall,
wherein, in a first configuration, the seal has a proximal position relative to the second distal wall, such that the seal inhibits fluid flow around the second distal wall, and
wherein, in a second configuration, the seal has a distal position relative to the second distal wall, such that the seal is configured to permit fluid flow around the second distal wall and into the first space.

2. The medical device of claim 1, further comprising a control member extending within the lumen of the sheath, wherein the control member is fixedly coupled to the first distal wall and extends distally relative to the first distal wall.

3. The medical device of claim 2, wherein, a diameter of a distal portion of the control member is greater than a diameter of a proximal portion of the control member, wherein the distal portion of the control member is distal of the first distal wall.

4. The medical device of claim 2, wherein the lumen of the sheath is a first lumen, and wherein the control member includes a second lumen extending within the control member from a proximal end to a distal end of the control member.

5. The medical device of claim 4, wherein the control member is configured to deliver a gas via the second lumen, and wherein, upon delivery of a fluid from the first lumen into the first space and a gas from the second lumen into the first space, the gas at least partially atomizes the fluid in the first space.

6. The medical device of claim 1, wherein the first distal wall includes one or more openings configured to permit fluid flow distally past the first distal wall.

7. The medical device of claim 1, wherein a gap is defined between a perimeter of the second distal wall and a wall defining the lumen of the sheath, wherein, in the second configuration, fluid is permitted to flow around the second distal wall via the gap.

8. The medical device of claim 1, wherein one or more spacers are disposed between the first distal wall and the second distal wall, wherein the one or more spacers are configured to define a fixed distance between the first distal wall and the second distal wall, and wherein the one or more spacers are disposed within a central opening of the seal.

9. The medical device of claim 1, wherein the seal is an O-ring.

10. The medical device of claim 1, further comprising a biasing member disposed proximally of the second distal wall.

11. The medical device of claim 10, wherein, in the first configuration, the biasing member is in an expanded configuration, and wherein, in the second configuration, the biasing member is in a contracted configuration.

12. The medical device of claim 10, further comprising:
a third distal wall fixed to a proximal end of the biasing member; and
a third space defined between the second distal wall and the third distal wall, wherein the biasing member is disposed within the third space.

13. The medical device of claim 12, further comprising a fourth distal wall proximal of the third distal wall, wherein a plurality of distal openings extends through the fourth distal wall, and
wherein, in the first configuration, a fourth space is defined between the third distal wall and the fourth distal wall, and wherein fluid is permitted to flow distally (i) into the fourth space via the plurality of distal openings, (ii) around the third distal wall, and (iii) into the second space.

14. The medical device of claim 13, wherein the fourth distal wall is sealed with respect to a wall defining the lumen of the sheath.

15. The medical device of claim 13, further comprising:
a fifth distal wall having a plurality of proximal openings extending through the fifth distal wall; and
a plurality of tubular members extending between the fourth distal wall and the fifth distal wall.

16. A medical device, comprising:
a handle having a stationary body and a movable body, wherein the movable body is movable relative to the stationary body;
a sheath fixed to a distal end of the handle, the sheath defining a first lumen extending from a proximal end to a distal end of the sheath;
a first distal wall disposed within the first lumen, wherein a first space is defined distally of the first distal wall;
a second distal wall disposed proximally of the first distal wall;
a seal disposed between the first distal wall and the second distal wall; and
a control member defining a second lumen extending from a proximal end to a distal end the control member, wherein a proximal end of the control member is fixed to the movable body and a distal end of the control member is fixed to the first distal wall,
wherein, in a first position of the movable body relative to the stationary body, the seal is configured to inhibit fluid flow around the second distal wall, and
wherein, in a second position of the movable body relative to the stationary body, and the seal is configured to permit fluid flow around the second distal wall and into the first space.

17. The medical device of claim 16, wherein the control member is configured to deliver a gas via the second lumen, and wherein, in the second position of the movable body, upon delivery of a gas from the second lumen into the first space, the gas at least partially atomizes a fluid within the first space.

18. The medical device of claim 16, wherein a gap is defined between a perimeter of the second distal wall and a wall defining the first lumen of the sheath, wherein, in the second position of the movable body relative to the stationary body, fluid is permitted to flow around the second distal wall via the gap.

19. A medical device, comprising:
a sheath having a proximal end, a distal end, and a lumen extending within the sheath from the proximal end to the distal end;
a wall disposed within the lumen of the sheath and having a plurality of openings extending therethrough;
a plate disposed within the lumen of the sheath distally of the wall; and
a biasing member coupled to the plate;
wherein, in a first configuration of the medical device, the biasing member exerts a distal force on the plate to permit fluid to flow distally from the plurality of openings and around the plate; and
wherein, in a second configuration of the medical device, the biasing member exerts a proximal force on the plate to inhibit fluid from flowing distally from the plurality of openings.

20. The medical device of claim 19, wherein, in the second configuration, the plate abuts a distal face of the wall.
